(19) United States
(12) Patent Application Publication
(10) Pub. No.: US 2018/0052145 A1
(43) Pub. Date: Feb. 22, 2018

(54) Wastewater Analyser Assembly

(71) Applicants: Keith Perkins, Sussex (GB); Christopher Charles Back, Kent (GB)

(72) Inventors: Keith Perkins, Sussex (GB); Christopher Charles Back, Kent (GB)

(21) Appl. No.: 15/555,175

(22) PCT Filed: Mar. 4, 2016
(86) PCT No.: PCT/GB2016/050569

§ 371 Date: Sep. 1, 2017

(30) Foreign Application Priority Data
Mar. 17, 2015 (GB) 1504461.3

(51) Int. Cl. G01N 33/18; G05D 7/06

(52) U.S. Cl. CPC G01N 33/182; G05D 7/0617

(57) ABSTRACT

A wastewater analyser assembly comprises an overflow reservoir for receiving wastewater from an input. The overflow reservoir comprises a weir for wastewater to overflow and exit the overflow reservoir. There is a wastewater sample outlet in the overflow reservoir for passing wastewater from within the overflow reservoir to an analyser.

### TECHNICAL FIELD

This disclosure relates to a wastewater analyser assembly and a method of analysing wastewater.

### BACKGROUND

Traditionally, wastewater is treated using chlorine as a disinfectant. Doses of chlorine above a certain level can have negative impacts on health and the environment. Therefore, it is necessary to ensure that the concentration of chlorine is reduced below an acceptable level before the wastewater is re-used or re-introduced into the environment.

Typically, wastewater is analysed to ensure that the concentration of chlorine is below 5 mg/L, in order to verify that the wastewater is safe. Inaccurate detection of concentrations above this level can lead to harmful effects.

Colorimetric, potentiometric and amperometric techniques can be used in wastewater analysers to measure concentrations of chlorine in wastewater. Amperometric techniques can be carried out using membrane units or direct cell units.

Direct cell units use a cell with two or three electrodes that are in direct contact with the sample as it is passed through the cell. The electrodes are connected to a control unit, which accepts a current from the electrodes and converts the current to a voltage. The voltage is output to an amplification circuit, which processes the signal via a processor hardware and software system.

Peracetic acid (PAA) can also be used as a disinfectant for wastewater. However, the acceptable level for PAA in wastewater is much less than 5 mg/L. For instance, in some applications it is required that the concentration of PAA in treated wastewater is below 0.1 mg/L. Current analysers for measuring concentrations of chlorine are incapable of measuring concentrations of PAA to the required level of accuracy.

The use of PAA in wastewater treatment presents a particular problem because it exposes the electrochemical cell and filter to contaminants, such as trachyderma, fungi and algae, which are produced in the process of treating sewage/wastewater. Trachyderma is a type of fungi which can develop relatively long spores. Whilst trachyderma is relatively harmless, the long spores can block the inside of the cell and the filter. This can lead to inaccurate readings from the analyser.

There is a need for wastewater analysers to measure the concentration of a contaminant, such as chlorine and/or PAA, in wastewater to a high level of accuracy.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

According to an aspect of the invention there is provided a wastewater analyser assembly comprising: an overflow reservoir for receiving wastewater from an input; the overflow reservoir comprising a weir for wastewater to overflow and exit the overflow reservoir; and a wastewater sample outlet in the overflow reservoir for passing wastewater from within the overflow reservoir to an analyser.

According to another aspect of the invention there is provided a method of analysing wastewater comprising: inputting wastewater into an overflow reservoir; the overflow reservoir comprising a weir for wastewater to overflow and exit the overflow reservoir; passing wastewater from within the overflow reservoir to an analyser, via a wastewater sample outlet in the overflow reservoir; and using the analyser to detect an amount of a contaminant in the wastewater.

According to another aspect of the invention there is provided a wastewater analyser assembly comprising: a first tube portion arranged to receive wastewater from an input tube within the first tube portion; a wastewater sample outlet in the first tube portion for passing wastewater from within the first tube portion to an analyser; and a flow rate controller having a cleaning flow rate mode arranged to provide a wastewater flow rate from the input tube which forces a jet of wastewater out of the first tube portion, thereby pulling fluid from the wastewater sample outlet into and out of the first tube portion.

According to an aspect of the present disclosure there is provided an amperometric analyser assembly for measuring the concentration of a species, the analyser assembly comprising: an electrochemical cell. Optionally, the electrochemical cell further comprises a plurality of glass or uPVC spheres. These beads may assist in the removal of dirt deposits from the electrodes of the cell and act to remove hydrogen bubbles produced in the process of passing a current between the two electrodes, thereby increasing the accuracy of the reading being taken.

Optionally, the assembly further comprises a constant head pipework assembly with an integrated venturi system. This assembly may give stable readings at low concentrations and may enable the removal of contaminant particles. The assembly may further comprise a small tank with a pump. This system is used to verify the accuracy of the analyser indication of concentration, due to the very low levels of concentrations. The assembly may comprise a filter fluidly connected to the constant head pipework, wherein at least part of the filter is arranged to pulsate in use. Pulsation of at least part of the filter inhibits the settlement and growth of fungi, algae and trachyderma.

The species, the concentration of which has to be determined, is preferably in solution, especially in solution in an aqueous medium. The assembly may be configured to determine the concentration of one or more oxidants, in particular of one or more peracids, such as peracetic acid.

The filter may be arranged to pulsate at a frequency of from about 0.1 to about 20 pulses per second, preferably from about 0.5 to about 10 pulses per second, more preferably from about 1 to about 5 pulses per second.

The frequency may be selected to obtain a constant flow of fluid through the chemical cell via the constant head pipework. The filter may comprise a flexible membrane, for example a nylon membrane which flexes when sufficient force is applied to it. In the context of the specification, it will be understood that the membrane may “flex”, be “flexible” or be subject to “flexing” insofar as the membrane has a degree of pliancy such that it will yield when force is applied to it, and such that it may return to its original shape, unless sufficient force is used to irreversibly damage the structural integrity of the membrane. The membrane is adjustable to change and is able to bend without breaking.

The flexible membrane may be arranged to pulsate on circulation of fluid through the filter. In the context of the specification, it will be understood that the fluid, or at least part of the filter, especially the membrane, may “pulse”, be “pulsed” or be subject to “pulsing” or “pulsation” insofar as the flow of fluid is oscillated in a rhythmical fashion, or insofar as at least part of the filter, especially the membrane, expands and contracts, or vibrates, rhythmically.

The resulting flow is mechanically connected by a series of valves to the constant head pipework which in turn provides a constant flow of fluid through the electrochemical cell and on selected occasions will produce a vacuum to clean the electrochemical cell (cell) and its associated pipework.

According to one example, the filter or filters may be a modified version of the type known in the art such as those described in GB 2293333. In contrast to the filter described in GB 2293333, the filter is provided with a flexible membrane against which fluid is sprayed to effect pulsation of the filter. According to this specific embodiment, the assembly may comprise a jet adapted to rotate. Said jet acts as a backwash for the filter and can spray or direct fluid into the filter, especially onto the membrane of the filter, thus causing at least part of the filter to pulsate. When fluid is sprayed or directed against the flexible membrane in the manner described above, the membrane flexes repeatedly, thereby creating a pulsing effect. Fluid can also be pulsed into the filter/onto the membrane, thus causing pulsation of the filter / membrane.

The analyser assembly comprises a housing which houses the cell, a microprocessor, optionally a timer for automation of the vacuum cleaning of the cell, a small tank and pump for verification of the unit operation, a constant head pipework assembly which also contains a configuration to produce a vacuum on the increasing of sample water flow to the constant head pipework and a two position “L” port valve mechanically connected to the cell, constant head pipework and verification pump. The design of the vacuum cleaning is specifically produced to pull the residual of the fungi, algae and trachyderma from the cell containing the electrodes and from the pipework connecting the constant head to the cell and send it to waste.

In another example, referred to as a verification system, the assembly comprises a small tank that contains a small pump and an “L” port valve, one side of the valve is connected to the small pump and the other side to the sample water from the constant head device, the common outlet is connected directly to the cell containing the electrodes and cleaning system. This verification system is used to pass a specific sample value through the cell which will indicate on the microprocessor the level of oxidant present in the specific sample, so verifying the analyser is operating accurately without the need of a second indication source.

The assembly typically comprises a microprocessor, which comprises a motherboard, the motherboard preferably comprising a power source and a signal processing board isolated there from and being a twin-set processing system.

The signal processing board and the power source are isolated in the sense that they are arranged so that the power source does not cause interference with the signal being processed. For example, the signal processing board and the power source can be placed far enough apart such that the signal does not experience any interference from the power source, or such that any such interference does not affect the accuracy of the signal being processed. A suitable distance to space the boards would be about 5 mm. In addition, a spacer board of, for example, 2 mm thickness, may be located between the component sides of each plug in board. This enables the more accurate detection of small concentrations (0 to 5 mg per litre) of oxidant.

In another example the power source and the signal processing board are located on separate circuit boards. When the power source and the signal processing board are located on separate circuit boards, the signal does not experience any interference from the power source, or any such interference is mitigated to the extent that it does not unduly diminish the accuracy of the signal being processed.

Optionally the assembly comprises a microprocessor controller comprising predetermined calibration data to calibrate the electrochemical cell. The microprocessor controller may be configured to impose a voltage on a sample of the species in order to reduce or cancel background signal or noise.

In one example, there is provided an amperometric method for measuring the concentration of a species, using the amperometric analyser assembly as described previously. This method is more accurate for analysing the concentration of oxidants, and in particular peracids such as peracetic acid, in solution. In particular, the method described enables the accurate measurement of peracetic acid concentration in the range 0 to 5 mg per litre.

Also, the assembly may be configured to determine the concentration of one or more peracids, typically peracetic acid. The method may comprise imposing voltage on a sample of the species in order to reduce or cancel background signal or noise. The method may comprise altering the zero referencing point of a signal amplifier circuit, the alteration comprising determining the amount of voltage to be imposed by measuring the positive and/or negative variation of voltage from zero; and imposing a voltage on a sample of an oxidant under measurement in order to reduce or cancel background signal or noise. This method is much simpler than the use of a reference electrode, and enables readings to be taken using an apparatus which requires much less maintenance than a reference electrode.

In one example, there is provided an amperometric analyser assembly for measuring the concentration of a species, the analyser assembly comprising: an electrochemical cell and a microprocessor which comprises a motherboard, the motherboard comprising a power source and a signal processing board isolated therefrom.

The signal processing board and the power source are isolated in the sense that they are arranged so that the power source does not cause interference with the signal being processed. For example, the signal processing board and the power source can be placed far enough apart such that the signal does not experience any interference from the power source, or such that any such interference does not affect the accuracy of the signal being processed. A suitable distance to space the boards would be about 5 mm. In addition, a spacer board of, for example, 2 mm thickness, may be located between the component sides of each plug in board. This enables the more accurate detection of small concentrations (0 to 25 mg per litre) of oxidant.

The power source and the signal processing board may be located on separate circuit boards. When the power source and the signal processing board are located on separate circuit boards, the signal does not experience any interference from the power source, or any such interference is mitigated to the extent that it does not unduly diminish the accuracy of the signal being processed.

In another example, there is provided an amperometric analyser assembly for measuring the concentration of a species, the analyser assembly comprising: an electrochemical cell and a microprocessor controller, the microprocessor controller comprising predetermined calibration data configured to calibrate the electrochemical cell.

In one example, the microprocessor controller receives a signal from the cell, and processes the signal equating the current to a lookup table. The lookup table may be produced by measuring the current produced in the analyser and equating this current to the peracetic acid reading shown by a separate calibration device, each current change is then referred to the lookup table and a correction factor is applied to the reading such that a corrected output reading is displayed on the analyser. The analyser may contain more than one lookup table, and may have several lookup tables produced using different calibration equipment. This has the effect to provide accurate and quick measurement of peracetic acid or of other oxidants, especially in aqueous solution.

In one example, there is provided an amperometric analyser assembly for measuring the concentration of a species, the analyser assembly comprising an electrochemical cell, the electrochemical cell comprising a plurality of glass or uPVC spheres. These beads may assist in the removal of dirt deposits from the electrodes and act to remove hydrogen bubbles produced in the process of passing a current between the two electrodes, combined with the constant head pipework containing within the assembly, a vacuum cleaning device produced by increasing the flow to the constant head system either manually or automatically via a timer system, greatly reduces the need to manually clean the electrodes. This increases the accuracy of the reading being taken, and minimises the need to manually clean the electrodes.

In one example, there is provided a verification system for passing a known concentration of oxidant through the measurement cell which displays the concentration on the microprocessor unit

In another example, there is provided an amperometric method for measuring the concentration of a species, using the amperometric analyser assembly described herein, the method comprising: imposing voltage on a sample of the species in order to reduce or cancel background signal or noise; and using the electrochemical cell to determine the concentration of the species therein.

In one example, the microprocessor controller can impose a voltage on a sample of the species in order to reduce or cancel background signal or noise. To enable the analyser to provide stable and reproducible readings, and to prevent drift at the zero calibration position, the voltage of the solution in the cell should be “null”. This is achieved by measuring a voltage produced by the cell, and then imposing this positive or negative voltage at the cell input. The magnitude of the voltage is measured across the cell input and the bias voltage is added to or subtracted from the circuit until a zero voltage is shown at the cell terminals. The zero is automatically compensated for using the cell biasing circuit input parameters.

In one example, the method comprises altering the zero referencing point of a signal amplifier circuit, the alteration comprising: determining the amount of voltage to be imposed by measuring the positive and/or negative variation of voltage from zero; and imposing a voltage on a sample of an oxidant under measurement in order to reduce or cancel background signal or noise. In one example, at least three of the above-mentioned aspects may be combined together.

In one example, the assembly of any of the previous aspects may be configured to determine the concentration of one or more oxidants, in particular of one or more peracids, such as peracetic acid, especially in solution, advantageously in an aqueous solution.

Optionally, the assembly of any of the previous aspects comprises a housing which houses the cell, a microprocessor, optionally a timer, a small tank for verification of the unit operation, a constant head pipework assembly which also contains a configuration to produce a vacuum on the increasing of the sample water flow. This design is specifically produced to pull the residual of the fungi, algae and trachyderma from the cell containing the electrodes and cleaning system.

The assembly may comprise a small tank that contains a small pump and an “L” port valve, one side of the valve being connected to the small pump and the other side to the sample water from the constant head device, the common outlet is connected directly to the cell containing the electrodes and cleaning system. This verification system is used to pass a specific sample value through the cell which will indicate on the microprocessor, the level of oxidant present in the specific sample, so verifying the analyser is operating accurately without the need of a second indication source. Optionally a fluid inlet and a fluid outlet are provided on the housing. The filter fluidly connected to the electrochemical cell is located outside the housing.

According to an aspect of the disclosure there is provided an amperometric analyser assembly for measuring the concentration of a species, the analyser assembly comprising: an electrochemical cell and at least one filter fluidly connected to the electrochemical cell, wherein at least part of the filter is arranged to pulsate in use.

The filter may comprise a flexible membrane. The flexible membrane may be arranged to pulsate at a frequency of from about 0.1 to about 20 pulses per second, preferably from about 0.5 to about 10 pulses per second, more preferably from about 1 to about 5 pulses per second.

The assembly may be configured to determine the concentration of one or more peracids, preferably of peracetic acid.

The assembly may comprise a housing which houses the cell, a microprocessor, optionally a timer for automation of the vacuum cleaning of the cell, a small tank and pump for verification of the unit operation, a constant head pipework assembly which also contains a configuration to produce a vacuum on the increasing of sample water flow to the constant head pipework and a two position “L” port valve mechanically connected to the cell, constant head pipework and verification pump.

The assembly may comprise a small tank that contains a small pump and an “L” port valve, one side of the valve is connected to the small pump and the other side to the sample water from the constant head device, the common outlet is connected directly to the cell containing the electrodes and cleaning system. This verification system is used to pass a specific sample value through the cell which will indicate on the microprocessor, the level of oxidant present in the specific sample, so verifying the analyser is operating accurately without the need of a second indication source.

Optionally, an actuated two way valve and a fluid inlet and a fluid return are provided on the housing. The actuated two way valve may be used to increase the sample flow to the constant head assembly for a timed operation and period of operation via the optional timer.

The assembly may further comprise a backwashing means for the filter, the backwashing means being arranged to direct fluid into the filter and cause the filter to pulsate.

The assembly may further comprise a microprocessor, which comprises a motherboard, the motherboard comprising a power source and a signal processing board isolated therefrom.

The assembly may further comprise a microprocessor controller, the microprocessor controller comprising predetermined calibration data configured to calibrate the electrochemical cell.

The microprocessor controller may be configured to impose a voltage on a sample of the species in order to reduce or cancel background signal or noise.

The electrochemical cell may further comprise a plurality of glass or uPVC spheres.

According to another aspect of the disclosure there is provided an amperometric method for measuring the concentration of a species, using the amperometric analyser assembly as described above, the method comprising: a sample pump with a filter, pulsating at least part of the filter assembly, a constant head device containing a venturi system and using the electrochemical cell to determine the concentration of the species therein and using a tank and pump with a calibrated sample to verify the analyser accuracy.

The method may comprise imposing voltage on a sample of the species in order to reduce or cancel background signal or noise.

The method may comprise altering the zero referencing point of a signal amplifier circuit, the alteration comprising: determining the amount of voltage to be imposed by measuring the positive and/or negative variation of voltage from zero; and imposing a voltage on a sample of an oxidant under measurement in order to reduce or cancel background signal or noise.

### BRIEF DESCRIPTION OF THE DRAWINGS

Embodiments of the invention will be described, by way of example, with reference to the following drawings, in which:

FIG. 1 shows an example of a wastewater analyser assembly in a first mode of operation;

FIG. 2 shows an example of the wastewater analyser assembly in a second mode of operation;

FIG. 3 shows another example of the wastewater analyser assembly in the first mode of operation;

FIG. 4 shows another example of the wastewater analyser assembly in the second mode of operation; and

FIG. 5 shows a sample bottle and holder for testing and calibrating the analyser assembly.

### DETAILED DESCRIPTION

Described below is an arrangement of ducts for a wastewater analyser where wastewater from an input flows into an overflow reservoir and over a weir, which creates a ‘constant head’ level at the weir. The wastewater flows out of a sample outlet in the overflow reservoir for transportation to an analyser.

There is a constant flow rate from the sample outlet to the analyser due to the ‘constant head’ level at the weir. This allows the wastewater to flow through the analyser at a constant rate. This helps to provide more accurate measurements.

The weir is particularly useful, for example, where there is a surge or a drop in flow rate through the input tube. Normally, this would lead to a change in flow rate through the analyser and an inaccurate reading in the detected concentration of a contaminant. However, in the present invention, the ‘constant head’ is maintained even if there is a surge or a drop in flow rate. This helps to prevent the analyser from producing inaccurate readings.

The assembly has a flow rate controller with an increased flow rate mode, or “cleaning flow rate mode”. In the increased flow rate mode a suction force is created that can clean the analyser by pulling fluid from the analyser through the sample outlet. This allows the analyser to be cleaned, without disassembling the wastewater analyser assembly.

Cleaning the analyser helps to provide more accurate measurements. This is particularly important in a wastewater analyser assembly, where peracetic acid (PAA) is used, because fungi and other debris are more likely to build up in the analyser cell, which can compromise its readings. The flow rate controller has a decreased flow rate mode, or “normal flow rate mode” where wastewater overflows the wall portion at the weir and the wastewater from the input exits the sample outlet due to gravity. In this way, the constant flow rate is provided through the analyser.

The assembly has a branch tube and a branch valve that allows a suction force to be activated simply by turning the branch valve to a closed position. The suction can be de-activated by turning the branch valve to an open position, which enables the constant flow rate through the analyser.

There is timer which can control the flow rate controller according to a pre-determined schedule. This allows the analyser to be cleaned automatically at predefined intervals. This helps to ensure that the analyser is kept clean.

There is also a manual actuator which a user can use to manually control the flow rate controller. In some situations, such as when the analyser is underperforming, it may be necessary to clean the analyser before the next automatic cleaning interval. The manual actuator allows a user to clean the analyser at any time.

The assembly has a sample selector valve which can selectively pass either wastewater from the overflow reservoir or a control sample to the analyser for testing. Thus, the analyser can be calibrated, verified and zeroed.

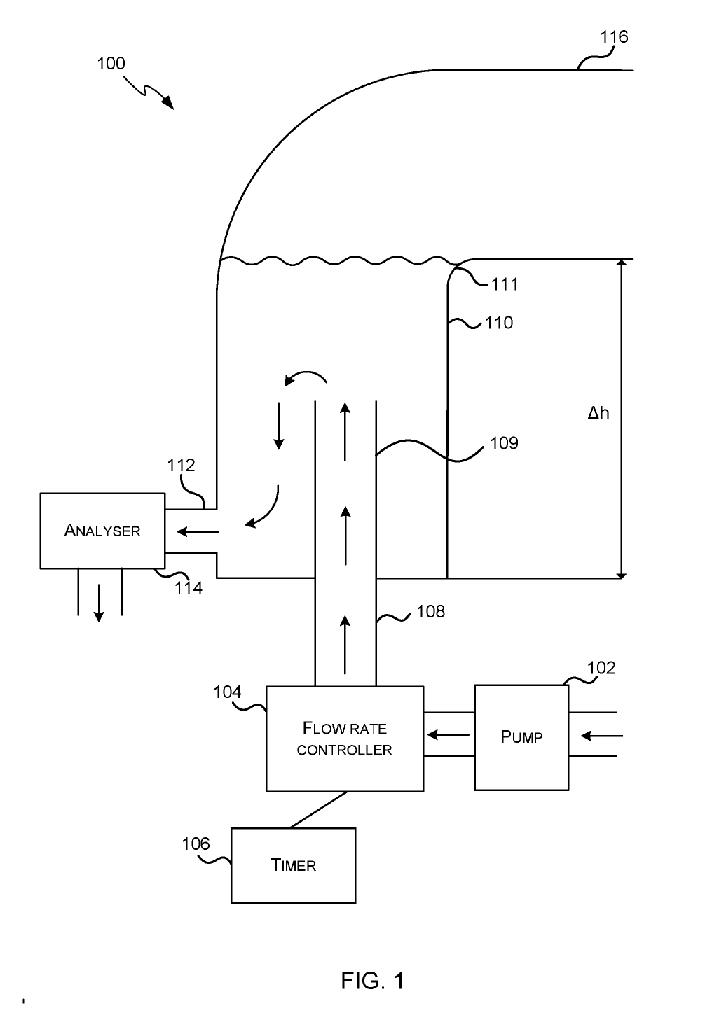

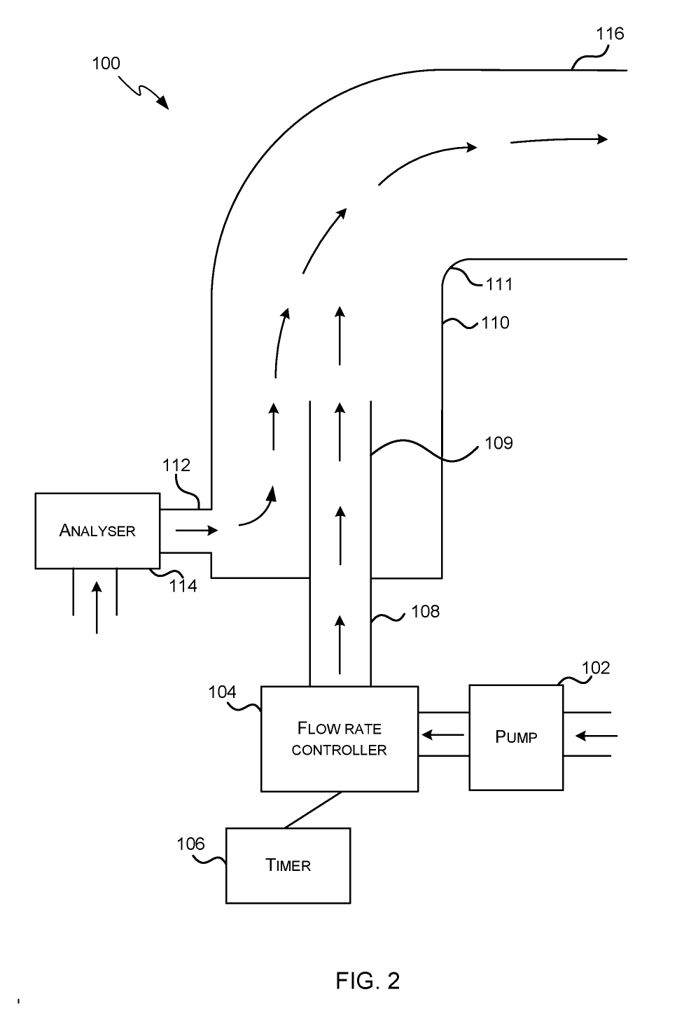

Referring to FIGS. 1 and 2, there is provided a wastewater analyser assembly 100 comprising a pump 102, a flow rate controller 104 connected to a timer 106, and an input tube 108 connected to an overflow reservoir 110. There is a wastewater sample outlet 112 in the overflow reservoir 110, which is connected to an analyser 114 for measuring the concentration of a contaminant in wastewater. It is important to maintain a constant flow rate through the analyser 114, in order for the analyser 114 to provide accurate readings. Furthermore, it is important for the analyser 114 to be kept clean and free of debris.

In a first mode of operation, as illustrated in FIG. 1, the pump 102 pumps treated wastewater from a reservoir into the input tube 108. The input tube 108 extends inside the overflow reservoir 110, forming an overflow chamber 109 within the overflow reservoir 109. Therefore, wastewater rises up the input tube 108 and overflows the overflow chamber 109, which acts as a weir. The overflow reservoir 110 fills with wastewater up to the peak of an overflow wall portion 111, which acts as another weir.

The overflow wall portion 111 maintains the wastewater at a constant head level Δh. This maintains a constant flow rate through the wastewater sample outlet 112 into the analyser 114. In the first mode of operation the flow rate controller 104 is in a decreased flow rate mode (or normal flow rate mode), where the magnitude of the flow rate is such that the weir is formed at the wall portion 111 and wastewater exits the overflow reservoir 110 due to gravity.

In a second mode of operation, as illustrated in FIG. 2, again the pump 102 pumps treated wastewater from a reservoir into the input tube 108. However, in the second mode the flow rate controller 104 is in an increased flow rate mode (or cleaning flow rate mode). Here, the magnitude of the flow rate forces a jet of wastewater out of the overflow chamber 109 and out of the overflow reservoir 110, and through a drain tube 116, which is connected to the end of the overflow reservoir 110. Here, the magnitude of the flow rate is such that the weir is not created at the overflow wall portion 111

The overflow chamber 109 has a reduced cross-sectional area with respect to the overflow reservoir 110. This feature coupled with the increased flow rate helps to create a pressure drop at the wastewater sample outlet 112. This pulls fluid through the analyser 114, through the sample outlet 112, and out through the drain tube 116. This helps to clean the analyser.

The timer 106 is arranged to activate the increased flow rate mode in accordance with a pre-determined schedule. This helps to clean the analyser 114 on a regular basis, without manual intervention. The flow rate controller 104 may comprise a manual actuator arranged to activate the increased flow rate mode. Thus, a user may clean the analyser whenever cleaning is required.

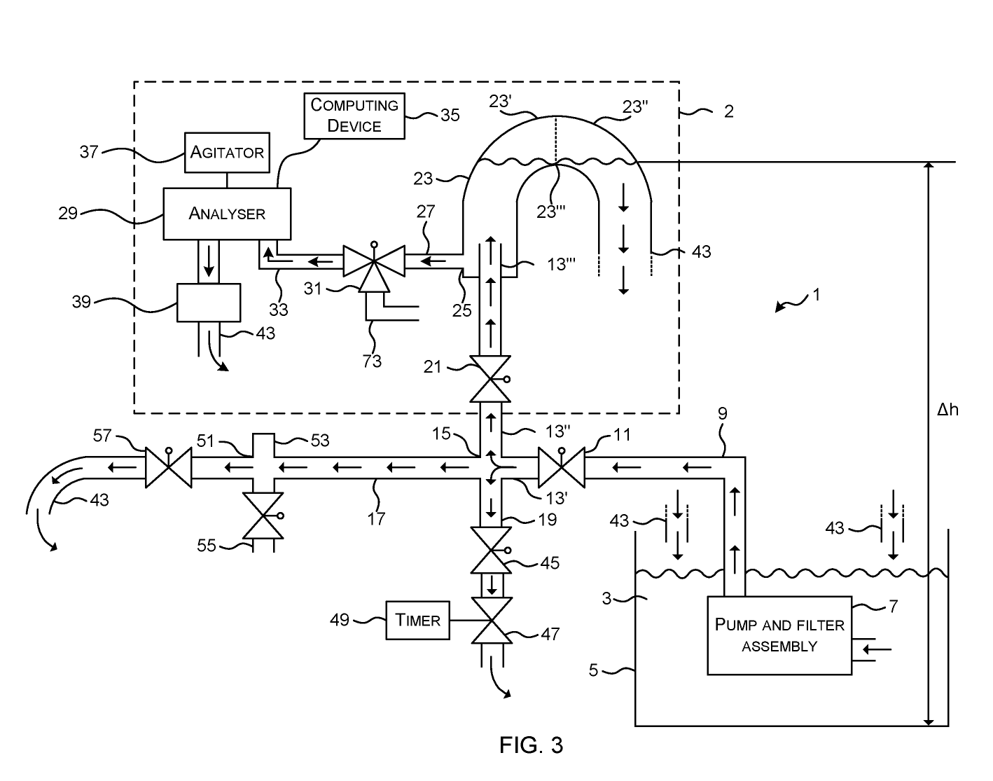

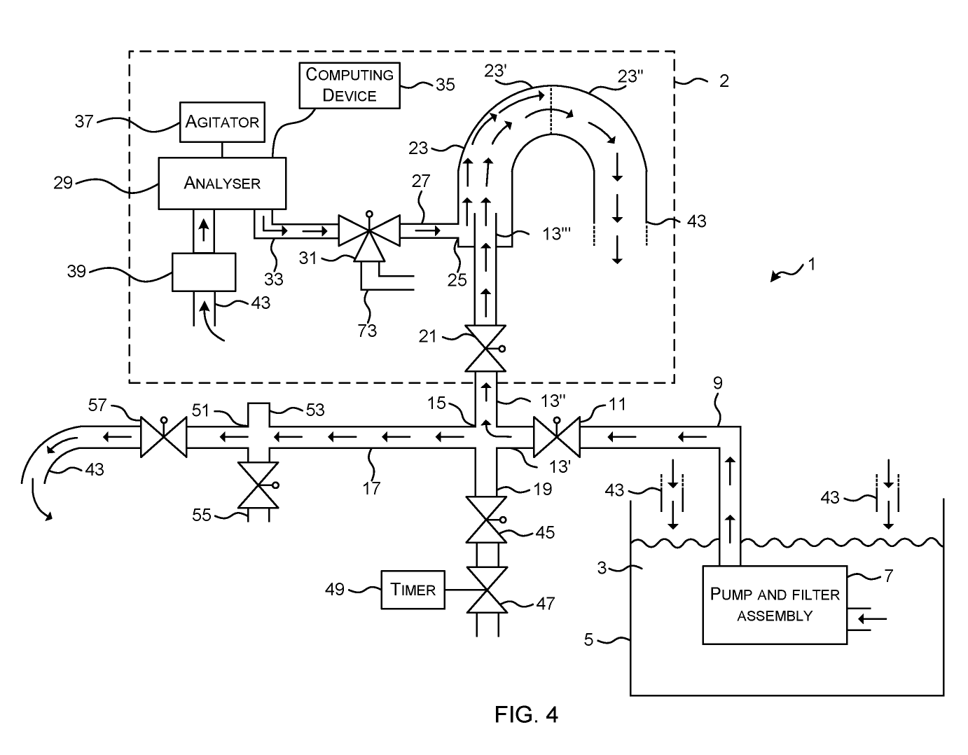

Referring to FIGS. 3 and 4, there is provided another example of the wastewater analyser assembly 1 partially contained within a housing 2. The wastewater analyser assembly 1 is arranged to measure the concentration of a contaminant in a volume of wastewater 3, held within a wastewater reservoir 5.

In this example, the analyser assembly 1 is arranged to measure the concentration of peracetic acid (PAA) in a volume of treated wastewater 3, in order to determine whether the wastewater 3 is safe for re-use or for re-introduction into the environment. It will be appreciated by those skilled in the art that the assembly may be used to measure concentrations of other substances, such as chlorine, or for any other appropriate application.

There is a pump and filter assembly 7 submerged within the wastewater 3 in the reservoir 5. The pump and filter assembly 7 may be one similar to that described in GB 2293333. The filter helps to prevent the pump and the analyser assembly 1 from becoming clogged with fungi and other debris. This is a particular problem when PAA is used to treat the wastewater 3. Any other suitable pump could be used, other than the pump described in GB 2293333. The pump may be used with another type of filter or without a filter.

The pump 7 forces wastewater 3 from the reservoir 7 and up into a first end of a reservoir tube 9. The reservoir tube 9 extends upwards and then bends through 90° to extend in a horizontal direction.

At a second end of the reservoir tube 9 there is a valve 11. When the valve 11 is in an open position, wastewater 3 is input into a first part of an input tube 13′ of the assembly 1. However, when the valve 11 is in a closed position, wastewater 3 is prevented from entering the input tube 13′. The valve 11 is usually in the open position during normal operation of the analyser assembly 1.

A first mode of operation will now be described with reference to FIG. 3. In this mode the valve 11 is open and wastewater 3 flows into the first part of the input tube 13′. Wastewater 3 flows along the first part of the input tube 13′ until it reaches a junction 15, which branches off into a second part of the input tube 13″, a throughput tube 17 and a branch tube 19.

The second part of the input tube 13″ is upstanding (i.e. it extends vertically upwards). The throughput tube 17 extends horizontally in the same direction as the first part of the input tube 13′. The branch tube 19 extends vertically downwards in the same direction as the second part of the input tube 13″. The pressure at the junction 15 forces wastewater 3 through each of the second part of the input 13″, the throughput tube 17 and the branch tube 19.

The wastewater 3 rises up the second part of the input tube 13″ and passes through a valve 21, which is normally open. The end of the input tube 13″ extends inside the base of an overflow reservoir 23, which forms an overflow chamber 13″′ inside the overflow reservoir 23.

The overflow reservoir 23 is formed from a tube comprising a first 90° elbow bend 23′ and a second 90° elbow bend 23″ connected together to form a bend that extends upwards and then back along the same direction as the first part of the input tube 13″. The peak of the bend forms a wall portion 23′″ which provides a weir over which the wastewater 3 flows. Thus, when wastewater from the input tube 13 fills the overflow reservoir 23 and overflows the weir a ‘constant head’ Δh is created. The overflowing wastewater 3 enters the second elbow bend 23″ and exits the assembly 1 via a drain tube 43, which flows back into the wastewater reservoir 5.

There is a wastewater sample outlet 25 in the overflow reservoir. In this example, the wastewater sample outlet 25 takes the form of a through hole in a side-wall of the overflow reservoir 23. The through-hole is positioned towards a base of the overflow reservoir 23.

The wastewater sample outlet 25 is connected to a wastewater sample tube 27 which carries wastewater 3 from the overflow reservoir 23 to an analyser 29 for measuring the concentration of PAA in the wastewater 3. The analyser 29 comprises an electrochemical cell which has two dissimilar metal electrodes (not shown) which may be shaped in a spiral or as a tube, and a copper disc or ring electrode (not shown). The electrodes are arranged such that their flat surfaces are face to face, in the case of the platinum wire and copper disc. In the case of the gold tube and copper disc, the gold electrode sits in the centre of the copper disc.

In use, sample fluid flows between the electrodes. The cell may be equipped with a pH electrode (not shown) and the constant flow level may be controlled using a valve. The cell is used to give an output dependant on the concentration of the contaminant and the conductivity of the wastewater. These signals are processed by a microprocessor and readings are displayed on a LCD display at a computing device 35.

The wastewater 3 flows from the sample outlet 25, through the tube 27 and into the analyser 29 at a constant flow rate due to the ‘constant head’ created by the weir. Therefore, any fluctuations in pressure made, for example, due to operating conditions at the pump, will not affect the flow rate through the analyser 29. This helps the analyser 29 to output more reliable and accurate results.

In this example, the input tube 13″ extends through the base of the overflow reservoir 23 to form the overflow chamber 13″′ having an open end providing a second weir. The height of the wall portion 23′″ is greater than that of the overflow chamber 13″′ with respect to the base. Both the wall portion 23′″ and the overflow chamber 13″′ extend vertically upwards. This arrangement helps to maintain a constant flow rate through the analyser 29, for instance even if the pump stops temporarily or if there is a surge of pressure from the pump.

Whilst the wastewater 3 is flowing up the input tube 13 it also flows down the branch tube 19. The branch tube 19 is provided with a manual valve 45 and an electrically operated valve 47. Either or both of these valves are used for controlling flow rate through the branch tube 47 and the input tube 13. The electrically operated valve 47 is connected to a timer 49, which opens and closes the valve 47 according to a predetermined schedule. This will be explained in more detail below.

In the first mode of operation the manual valve 45 and the electrically operated valve 47 are both open. The flow through the branch tube 19 helps to maintain the flow rate through the input tube at the desired level, thus maintaining the constant head.

Whilst the wastewater 3 is flowing up the input tube 13 it also flows horizontally through the throughput tube 17. The throughput tube 17 is provided with a junction 51 which is closed off at one end 53 extending vertically upwards. There is an outlet at another end 55 which extends vertically downwards. This end 55 is provided with a valve which is normally closed. However, this valve can be open to aid in flushing wastewater from the assembly 1.

The junction 51 has another outlet which extends horizontally in the same direction as as the throughput tube. This outlet is provided with a valve 57 which is normally open. When the valve 57 is open, wastewater flows through it and into a drain tube 43 which leads to the reservoir 5. The arrangement of the throughput tube helps to ensure the desired flow rate out of the input tube 13.

The wastewater 3 from the overflow reservoir 23 flows along the wastewater sample tube 27 until it reaches a sample selector valve 31, which is normally open. The wastewater 3 passes through the valve 31 into an analyser tube 33, which feeds the analyser 29.

In this example, the analyser 29 is an amperometric analyser for detecting the concentration of PAA in the wastewater 3. However, another type of analyser for detecting the concentration of a contaminant may be used. For example, a potentiometric analyser for detecting the concentration of chlorine in wastewater 3 may be used.

The analyser 29 is connected to the computing device 35 comprising a processor which amplifies signals received from the analyser 29. The computing device 35 outputs an indicator of the concentration of PAA via a visual display. The computing device 35 may be arranged to trigger an alarm if the concentration rises above a threshold level, such as 0.1 mg/L.

There is also an agitator 37 connected to the analyser 29. The agitator 37 mechanically agitates the analyser 29, in order to clean the electrodes or cell in the analyser 29. This helps to dislodge the built up of hydrogen bubbles, which affect readings from the analyser. This also helps to clean debris such as fungi, which may build up in the analyser 29. In this example, the agitator 37 comprises a mechanical stirrer which stirs a plurality of uPVC or glass balls inside the analyser 29, between the electrodes through which the wastewater 3 passes.

The wastewater 3 exits the analyser via a translucent or transparent chamber 39. A user can use the chamber 39 as a visual aid in order to determine whether wastewater 3 is passing through the analyser 29. The wastewater 3 exits the analyser assembly 1 via a drain tube 43 which returns to the wastewater reservoir 5.

A second mode of operation will now be described with reference to FIG. 4. The set-up of the analyser assembly 1 in the second mode of operation is the same as described above, except either the manual valve 45 or the electrically operated valve 47 is closed.

In one example of the second mode of operation, a user closes the manual valve 45 using a manual actuator. This closes the branch tube 19 which increases the flow rate through the input tube 13. The flow rate through the input tube 13 increases such that a jet of wastewater 3 is forced out of end of the overflow chamber 13″′, and travels through the overflow reservoir 23 without creating a weir and the constant head level.

The overflow chamber 13′″ part of the input tube 13 has a reduced diameter in comparison with the overflow reservoir 23. Therefore, when the jet of wastewater exits the input tube 13, the pressure drops at the sample outlet to a negative pressure, or a vacuum. This is created due to the ‘venturi’ effect.

In another example, the overflow chamber 13″′ may have a reduced cross-sectional area in comparison to the cross-sectional area of the second part of the input tube 13″. This may increase the pressure of the vacuum at the wastewater sample outlet 25. In another example, there is a constriction in the overflow reservoir 23 at a point in the wastewater flow path after the overflow chamber 13″′. This constriction may be provided by the bend.

The vacuum pulls water from the analyser 29, through the wastewater sample outlet 25, and out through the drain tube 43. This may also suck fungi and other debris out of the analyser 29. This helps to clean the analyser cell without having to disassemble the analyser, which would render the analyser operable for a period of time. The drain 43 at the analyser 29 side is at least partially open to air, so that the vacuum can suck air through the analyser 29. This may clean the analyser 29 more effectively.

The manual valve 45 allows the analyser 29 to be cleaned at any given moment. For example, when the analyser is underperforming, the manual valve 45 could be closed to initiate cleaning in order to return the analyser assembly to its normal level of operating performance.

In another example of the second mode of operation, the electrically operated valve 47 is closed for a brief period of time in response to a signal sent from the timer 49. In this example, the timer 49 is arranged to close and open the valve 47 in accordance with a predetermined schedule. For instance, the timer 49 may be arranged to close the valve 47 once a day for ten seconds, during the evening. This helps to clean the analyser regularly, without manual intervention. This helps the analyser to output consistently accurate results.

In one example of the assembly described above, the reservoir tube 9, the first part of the input tube 13′, the second part of the input tube 13″, the overflow reservoir 23, the branch tube 19 and the throughput tube 17 all have the same diameter. In another example each of these sections has a diameter of 25.4 mm (1 inch). The diameter of the drain tube 43 may be, for example, 12.7 mm (½ inch).

In another example the overflow chamber 13″′ has a smaller diameter than the second part of the input tube 13″, for instance, the diameter of the overflow chamber 13″′ may be 9.53 mm (⅜ inch). In a further example, the diameter of the second part of the input tube 13″ is reduced at the outlet of the valve 21, for instance, this part may have a diameter of 12.7 mm (½ inch).

The dimensions described above are given as examples only in order to provide some context, and any other dimensions could be used instead. In addition, the term “tube” described above has been used to refer to any type of fluid duct, which may take any shape or size.

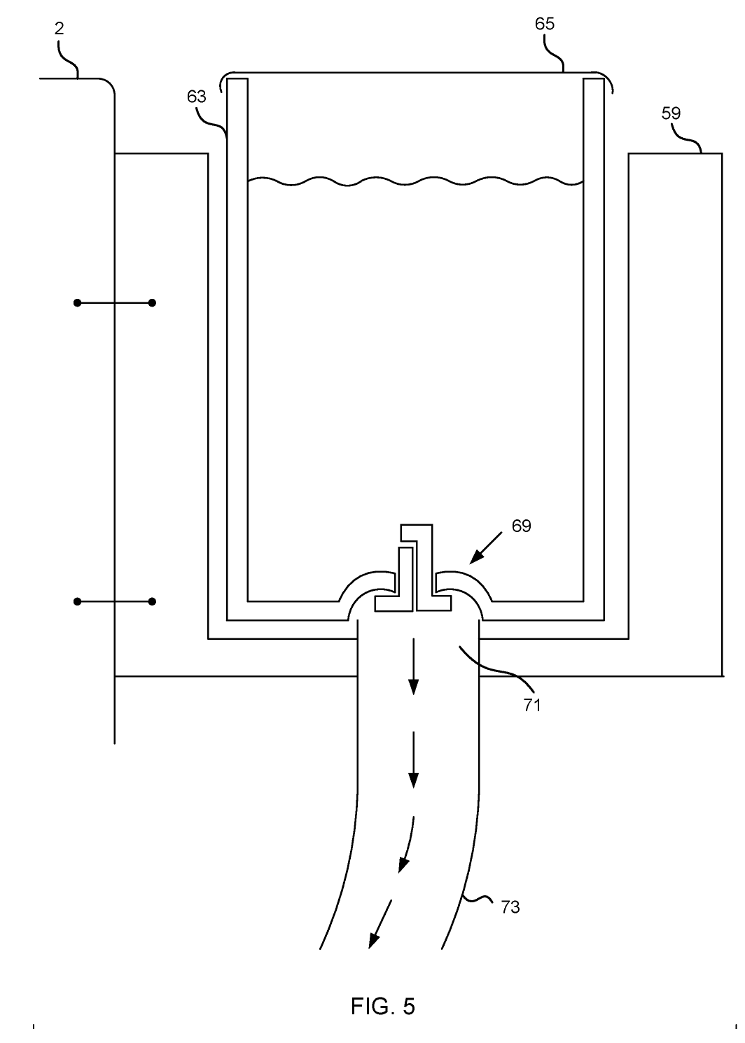

Referring to FIG. 5, there is a sample bottle holder 59 attached to the housing 2 by a series of screws. The sample bottle holder 59 is arranged to receive a detachable sample bottle 63. FIG. 5 illustrates the detachable sample bottle 63 within the holder 59, provided with a cap 65.

The sample bottle 63 is used to collect and store a liquid sample which can be used to test the analyser 29 or the sample within the bottle 63. The sample bottle 63 comprises a leak protection device 69 which closes the base of the bottle 63 when it is not attached to the holder 59. However, when the bottle 63 is placed inside the holder, the leak protection device 69 disengages. This allows the liquid in the bottle 63 to flow out of the leak protection device 69, and through the base.

There is a sample tube inlet 71 attached to the base of the holder 59, which is connected to a sample tube 73. The sample tube 73 leads to the sample selector valve 31, which is shown in FIGS. 3 and 4. The sample selector valve 31 can be turned so that the analyser receives either wastewater from the overflow reservoir 23 or from the sample bottle 63.

The sample bottle holder 59 is located at a greater vertical height than the analyser. Therefore, the liquid in the bottle 63 can flow directly to the analyser 29 without the need for a pump. However, it will be appreciated that a pump could be used to help transport a sample to the analyser 29, with the sample bottle 63 located above or below the analyser 29.

The sample bottle holder 59 may be used for a variety of applications. In one example, the sample bottle 63 contains a liquid that has been analysed elsewhere to confirm that it contains no PAA. When the bottle 63 is attached to the sample holder 59, the liquid passes through the sample tube 73, through the sample selector valve 31 and into the analyser 29. In this situation, the analyser 29 should confirm that 0 mg/L of PAA is in the sample. This may confirm that the analyser 29 is working properly. On the other hand, if the analyser does not confirm a concentration of 0 mg/L, the analyser can be calibrated accordingly (i.e. zeroed). This method could be used to test and calibrate for other concentrations of PAA, chlorine or another contaminant.

In another example, the analyser assembly 1 is being used to analyse water at one point in a water treatment system, which has a number of stages. In this example, a sample of wastewater from one of the stages is collected using the sample bottle 63. The sample bottle 63 is then placed in the holder 59, as described above. Here, the holder 59 and bottle 63 can be used to measure the concentration of a contaminant in wastewater from different stages in the system. Therefore, it is not necessary to provide an analyser assembly at each stage in the system.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method blocks or elements identified, but that such blocks or elements do not comprise an exclusive list and a method or apparatus may contain additional blocks or elements.

It will be understood that the above description of a preferred embodiment is given by way of example only and that various modifications may be made by those skilled in the art. Although various embodiments have been described above with a certain degree of particularity, or with reference to one or more individual embodiments, those skilled in the art could make numerous alterations to the disclosed embodiments without departing from the scope of this invention.

1. A wastewater analyser assembly comprising:
an overflow reservoir for receiving wastewater from an input;
the overflow reservoir comprising a weir for wastewater to overflow and exit the overflow reservoir;
and
a wastewater sample outlet in the overflow reservoir for passing wastewater from within the overflow reservoir to an analyser.

2. A wastewater analyser assembly according to claim 1 wherein the input comprises an input tube having an end point within the overflow reservoir;
the input tube having a smaller cross-sectional area than the overflow reservoir at the end point of the input tube.

3. A wastewater analyser assembly according to claim 1 comprising a flow rate controller having a cleaning flow rate mode arranged to provide a wastewater flow rate from the input which forces a jet of wastewater out of the overflow reservoir, thereby pulling fluid from the wastewater sample outlet.

4. A wastewater analyser assembly according to claim 3 wherein the flow rate controller comprises:
a branch tube, branched away from the input; and
a branch valve having an closed position for controlling the flow rate through the branch tube and the input;
wherein when the branch valve is in the closed position the flow rate controller is in the cleaning flow rate mode.

5. A wastewater analyser assembly according to claim 3 further comprising a timer arranged to activate the cleaning flow rate mode in accordance with a pre-determined schedule.

6. A wastewater analyser assembly according to claim 3 further comprising a manual actuator arranged to activate the cleaning flow rate mode.

7. A wastewater analyser assembly according to claim 1 comprising a flow rate controller having a normal flow rate mode arranged to provide a wastewater flow rate through the input, such that wastewater flows from the input and out of the wastewater sample outlet, and over the weir.

8. A wastewater analyser assembly according to claim 7 wherein the flow rate controller comprises:
a branch tube, branched away from the input; and
a branch valve having an open position for controlling the flow rate through the branch tube and the input;
when the branch valve is in the open position the flow rate controller is in the normal flow rate mode.

9. A wastewater analyser according to claim 1 wherein the input comprises an input tube connected to an overflow chamber within the overflow reservoir, the overflow chamber having an open end providing a second weir.

10. A wastewater analyser according to claim 9 wherein a portion of the input tube extends into the overflow reservoir thereby providing the overflow chamber.

11. A wastewater analyser according to claim 9 wherein the overflow reservoir comprises a base and the weir extends further from the base than the overflow chamber.

12. A wastewater analyser assembly according to claim 1 further comprising:
a control sample tube comprising a control sample inlet, for receiving a control sample, and a control sample outlet, for providing the control sample to the analyser;
a sample selector valve connected to the wastewater sample outlet, the control sample outlet and an analyser outlet, for providing the control sample or the wastewater to the analyser via the analyser outlet,
the sample selector valve having:
a first position arranged to pass wastewater from the overflow reservoir to the analyser; and
a second position arranged to pass the control sample from the control sample tube to the analyser.

13. A wastewater analyser assembly according to claim 1 further comprising an analyser arranged to detect an amount of a contaminant in the wastewater.

14. A wastewater analyser assembly according to claim 13 wherein the analyser is arranged to detect an amount of peracetic acid.

15. A wastewater analyser assembly according to claim 13 further comprising a mechanical agitator arranged to clean the analyser.

16. A wastewater analyser assembly according to claim 1 further comprising:
a throughput tube branched away from the input for reducing wastewater flow rate into the overflow reservoir.

17. A method of analysing wastewater comprising:
inputting wastewater into an overflow reservoir;
the overflow reservoir comprising a weir for wastewater to overflow and exit the overflow reservoir;
passing wastewater from within the overflow reservoir to an analyser, via a wastewater sample outlet in the overflow reservoir; and
using the analyser to detect an amount of a contaminant in the wastewater.

18. A method of analysing wastewater according to claim 17 wherein the input comprises an input tube having an end point within the overflow reservoir; the input tube having a smaller cross-sectional area than the overflow reservoir at the end point of the input tube.

19-22. (canceled)

23. A method of analysing wastewater according to claim 17 further comprising:
selecting either a control sample or wastewater from the overflow reservoir for provision to the analyser via an analyser outlet, using a sample selector valve having:
a first position arranged to pass wastewater from the overflow reservoir to the analyser; and
a second position arranged to pass the control sample to the analyser.

24. A method according to claim 17 wherein the analyser is arranged to detect an amount of peracetic acid; and the method further comprises:
using the analyser to detect an amount of peracetic acid.

25. (canceled)
